(19) United States
(12) Patent Application Publication
(10) Pub. No.: US 2009/0281510 A1
(43) Pub. Date: Nov. 12, 2009

(54) COLLECTION BAG FOR MALE INCONTINENCE

(76) Inventor: Harry Fisher, Jerusalem (IL)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/176,961

(22) Filed: Jul. 21, 2008

Related U.S. Application Data
(60) Provisional application No. 61/052,086, filed on May 9, 2008.

(51) Int. Cl. A61F 5/44

(52) U.S. Cl. 604/349; 604/351

(57) ABSTRACT

A collection bag for male incontinence covers a portion of the penis. The collection bag has flexible side walls forming a fluid container for holding urine. A mouth to the bag receives the penis and possibly the scrotum. The bag has conventional handles like a shopping bag. Connected to the handles and/or around the bag neck there is an anchoring for engaging the body. This can be a removable string to surround the body torso.

### RELATED APPLICATION

This application claims the benefit of and priority to U.S. Provisional Application Ser. No. 61/052,086, filed May 9, 2008, the contents of which are incorporated by reference herein in its entirety.

### BACKGROUND

This disclosure relates to male incontinence. More specifically, it concerns a urine collection bag for improved incontinence management. The system is essentially for use in bed and offers convenience and an increased freedom of movement.

This disclosure addresses disadvantages of prior art collection bags and systems. This disclosure also relates to a moisture management fabric composite, and more specifically, to a moisture management incontinence pad designed for men.

Presently available incontinence systems and pads are generally relatively more expensive and reusable.

### SUMMARY

The disclosure provides a urine collection bag for use in male incontinence management. There is a urine collection bag which can be worn in bed, for instance, at night relatively comfortably and under clothing. The bag is of a thin moisture proof material and is therefore comfortable to be used against the body. The bag is for easy single use and can simply be discarded after use.

A collection bag for male incontinence covers at least a portion of the penis. The collection bag has walls which can be first and second opposing flexible side walls joined together and forms a fluid container for holding urine. Between the first and second side walls of the bag, there is a mouth for receiving the penis and possibly the scrotum. The bag has conventional handles like a shopping bag, and as such is a thin pvc material shopping type grocery plastic bag. The mouth for the bag is formed in the area of the handles which are in the form of loops of material integrally formed and joined to the bag walls or are a cut out portion of the side walls.

Connected to the loop formed handles and/or around the bag mouth or neck there is an anchoring for engaging the body, and locating the bag on the body. This can be a removable string, or belt to surround the lower body torso.

The mouth of the bag can be normally large, and the loop formed handles can be tied in a relatively loose bow or knot thereby to close the mouth size so that there is a relatively tighter fit about the penis and/or scrotum. The free ends of the knotted loop handles are then used in the sense that the string, cord or belt is passed through the open loops and the bag is thereby tied about the stomach area of the torso.

Urine enters the bag and is absorbed by a multi-layer moisture fabric composite which includes a three-dimensional fabric composite. The liquid impermeable bag surrounds the absorbent layers. The fabric composite is for trapping urine moisture in the absorbent layers.

The urine management system provides an effective easy and inexpensive system for re-using or using disposable thin shopping type grocery holding bags and by providing a multi-layer moisture management fabric composite. The fabric composite includes a three-dimensional fabric and a plurality of moisture-absorbent layers for receiving and dispersing moisture such as urine entering the fabric composite.

The absorbent material can include a fine mesh fabric, and/or moisture wicking fibers for promoting moisture flow by capillary wicking. The moisture wicking fibers can be selected from a group consisting of polyester, nylon, and acrylic and can include antimicrobial fibers, yarns, cotton and wool, hydrophillic fibers such as cotton, felt, and wool.

The disposable pad includes the moisture absorbing components. The moisture management incontinence pad can be provided separately or be provided for use with special disposable bags.

The pad can utilize multiple components or layers, and for instance there can be a first wicking layer at a urine entry zone and then multiple layers of different absorbent fabric or materials.

The moisture management incontinence pad can combine many different fibers in any one desired construction, using the particular properties of each fiber. The moisture management incontinence pad is relatively light and comfortable and disposable.

The disclosure includes a urine management system wherein a package or kit is prepared for use. The kit would include multiple bags as defined with multiple absorbent pads and a suitable cord, belt or fastening device provided as part of that kit.

### DRAWINGS

Different features of the disclosure will become more apparent with reference to the description in conjunction with the accompanying drawings where like reference numerals denote like elements. These and other objects of the present disclosure are achieved in the embodiments. Some of these objects of the disclosure are set out. Other features of the disclosure will appear from the description in conjunction with the following drawings, in which:

FIG. 1 is a side view of the collection bag in use and showing part of the male torso.

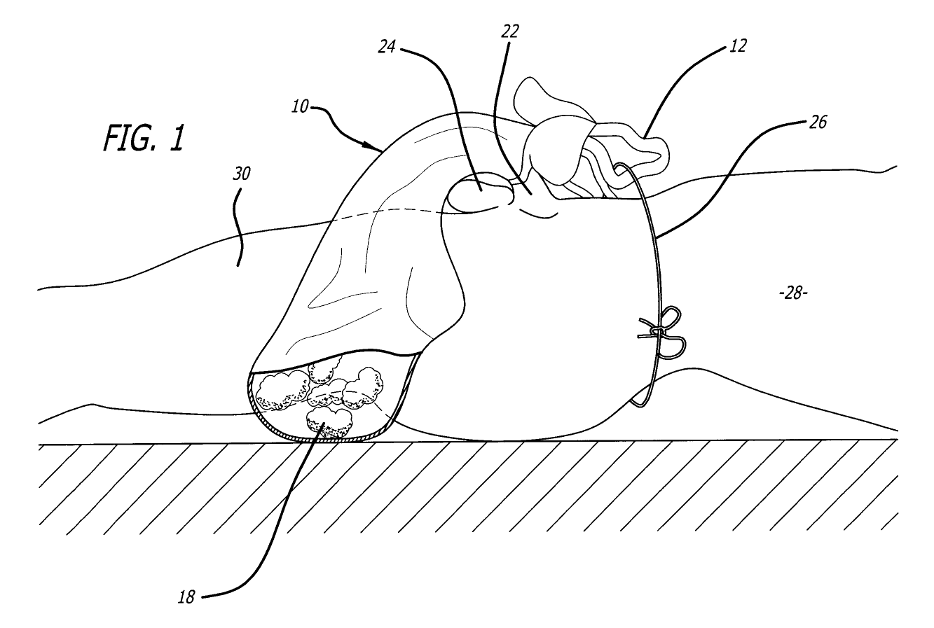

FIG. 2 is a front view of the collection bag in use and showing part of the male torso.

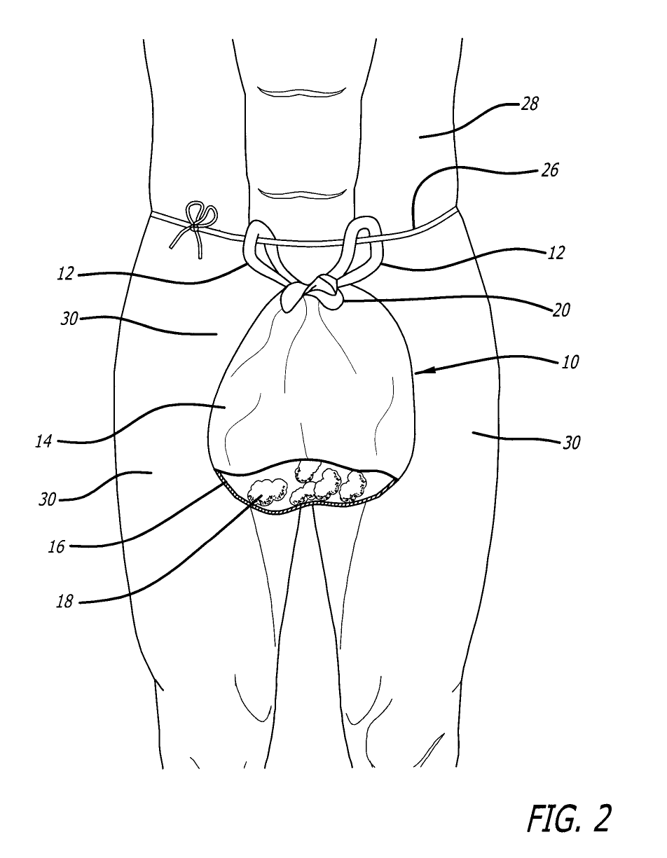

FIG. 3 is a representation of a kit having the bags, pads, and tying cord.

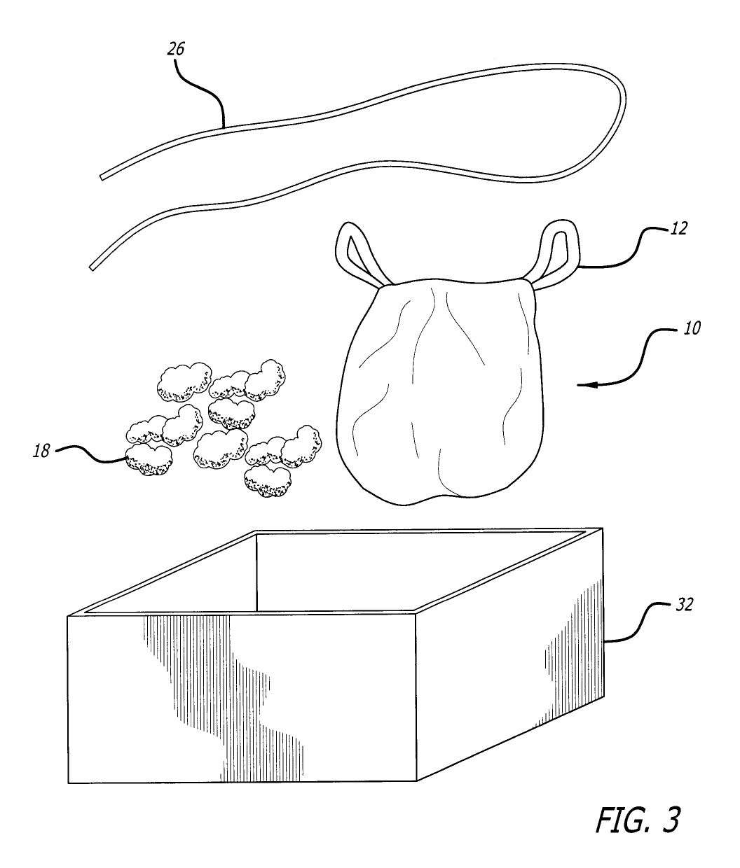

### DETAILED DESCRIPTION

The following embodiments according to the disclosure are given as an example only, without being limiting in any way, with reference to the accompanying drawings.

A urine collection bag is shown generally as reference numeral 10. The collection bag 10 is applicable for use in an incontinence management system for men especially when sleeping.

The bag 10 can be a conventional shopping bag with a pair of handles 12. The collection bag 10 is constructed of first and second opposing flexible side walls 14 and 16 joined together and forming a fluid container 10 for holding urine.

The side walls 14, 16 can be formed in a single mold and joined together in a conventional molding manner or heat sealed. Any other form of manufacture can be used so that it is an integral side wall system. The walls may be formed of a semi-transparent, white or skin tone polymer or may be opaque. The thickness of each side wall 14, 16 is about 8 mil. The capacity of bag 10 for the fluid is such that is can hold one or more absorbent pads 18, and the pads can absorb about 500 ml (cc).

Between the bag 10 first and second side walls 14 and 16, a mouth 20 is defined for receiving the penis 22 and possibly the scrotum 24. The bag 10 has conventional handles 12 like a shopping bag, and as such is a thin pvc material.

Connected to the loop formed handles 12 and/or around the bag neck there is an anchoring 26 for engaging the body 28. This can be a removable string 26, or belt to surround the body torso.

The mouth 20 of the bag 10 can be normally large, and the loop formed handles 12 can be tied in a relatively loose bow or knot 30 thereby to close the size of mouth 20 so that there is a relatively tighter fit about the penis 22 and/or scrotum 24. The looped sections of the knotted loop handles 12 are then used in the sense that the string, cord or belt 26 is passed through the loops 12 and the bag 10 is thereby tied about the torso.

Urine enters the bag 10 and is absorbed by a multi-layer pads 18 which are moisture fabric composites which includes a three-dimensional fabric composite. The liquid impermeable bag surrounds around the absorbent layers. The fabric composite is for trapping urine moisture in the absorbent layers.

A collection bag 10 provides for communicating with a penis and in some cases the scrotum and is for accommodating a portion of the penis and in some cases the scrotum.

The bag 10 can be formed of a conventional pvc or other synthetic resinous material or of a flexible elastomeric, and is essentially easily crushable and compactable so as to permit for comfortable placement with the body between the thighs 30 of a user.

When in use, urine leakage enters into the collection bag 10 where the urine is temporarily stored in a pad prior to disposal of the bag. Both the collection bag 10 and the pad may be conveniently disposed of after use. When the user awakes, and/or the bag 10 has been used and/or is full, and/or the one or more pads 18 are saturated, the bag 10 is removed form the body 28 by releasing the string or strap 26 around the torso. The pads are conveniently discharged into a toilet and the bag 10 discarded.

As such this is a reusable function for a shopping bag 10 which might otherwise be single use.

In some situations the bag 10 can be purpose made and the strap can be special formed. In other cases a belt can be used to encircle the body and pass through the handles on the shopping bag.

The moisture management incontinence pad 18 is for use in the bag 10 for men and is especially useful for wear at night to manage moderate to heavy urine leakage while sleeping. The incontinence pad 18 is for location loosely inside the bag 10. Alternatively there can be a non-purpose made reusable pad 18 rendered available for the bag 10.

In some cases there can be suitable fasteners such as snaps, hook and loop fasteners, or the like for use to locate the moisture absorbing pad 18 in the bag 10. The fasteners of the incontinence pad 18 can position and retain the pad 18 inside the bag 10.

In other cases the moisture absorbing pad 18 might just be absorbent paper such as paper towel, which can be folded otherwise located in the bag 10.

The incontinence pad 18 can be constructed of a multi-layer moisture management fabric composite. The fabric composite can include a dimensional fabric spacer, a wicking layer, and moisture-absorbent layers. The fabric composite or yarns can include moisture-wicking fibers, such as acrylic and polyester, which are made to promote the transport of moisture by capillary wicking.

These yarns can be constructed of high moisture-absorbent fibers, such as cotton and wool, and/or wicking fibers, such as polyester and acrylic, which operate to draw moisture inwardly and disperse it over the entire surface area of the pad. There can be breathable, open mesh fabric which promotes air circulation in the pad. The moisture-absorbent fiber can swell in excess of 20% when wet.

The wicking layer can cooperate with the moisture-absorbent layers to further move moisture as needed. The wicking layer can engage the wet fibers, and through capillary action further transports moisture as needed to highly absorbent layers where the moisture is dispersed. The wicking layer can include substantially wicking fibers, such as polyester and acrylic. The moisture-absorbent layers can comprise cotton, wool, and felt which swell up to 45% when wet.

Urine enters the bag 10 and then the incontinence pads 18. There can be a material around the absorbing layer of the pads 18 to deter liquid movement outwardly from the material that absorbed urine leakage in the incontinence pad 18. The fabric composite during the leakage of urine passes the moisture into the pad 18.

A urine collection bag 10 for a male incontinence device is described. This can include one or more pads 18, and the system includes a tying cord 26. The system can be packaged and marketed as an incontinence control kit 32 having several bags 10, pads 18 and a cord 26.

A multi-layer moisture management fabric composite is described and various details of the disclosure may be changed without departing from the scope of the disclosure.

Many details of the disclosure may be changed without departing from its scope. The description of the disclosure are provided for the purpose of illustration only and not for limitation.

Many other combinations of components or methods of describing the present disclosure are possible. For instance, if needed there can be bag with a deodorizing agent.

In other cases, there can be a shape-retaining male receptacle such as a male condom, for receiving the penis, and facilitating directing the penis into the mouth of the bag. In other variations there can be a Velcro system used for closing the open ends of the string, cord etc about the body or for securing the bag to the body or clothing worn by the user.

The disclosure is not limited to the embodiments described as an example and represented in the accompanying figures. Many different variations in size and scope and features are possible.

A person of ordinary skill in the art will recognize that many further combinations and permutations of the present disclosure are possible. The disclosure embraces all such alterations, combinations, modifications, and variations that fall within the spirit and scope of the appended claims. The disclosure includes any and all embodiments of the following claims.

1. A collection bag for male incontinence comprising:
flexible side walls to form a fluid container for holding urine;
between the walls there is a mouth defined for receiving the penis and possibly the scrotum;
handles for the bag; and
connected to the handles and/or around the bag neck there is an anchoring for engaging the body.

2. A collection bag as claimed in claim 1, including a cord to surround the body torso, the cord being openable and fastenable, and selectively removable from the bag handles.

3. A collection bag as claimed in claim 1, including a pad wherein when urine enters the bag, the urine is absorbed by the pad, the pad including a moisture absorbing fabric composite.

4. A collection bag as claimed in claim 3, wherein the pad includes multiple layers and includes a three-dimensional fabric composite.

5. A collection bag as claimed in claim 3, wherein the bag is liquid impermeable and surrounds the absorbent layers.

6. A collection bag as claimed in claim 3, wherein the fabric composite is for trapping urine moisture in absorbent layers.

7. A collection bag for use as a male incontinence containment device comprising:
a flexible bag with walls forming a fluid container for holding urine;
a bag neck formed with the walls and defining a mouth for receiving urine passed in to the bag; and
openings in the bag through which an elongated member can pass for tying the bag about the torso.

8. A collection bag as claimed in claim 7, wherein the bag is a thin flexible material and the openings are from loop shaped handles which normally extend from above the bag side walls.

9. A collection bag according to claim 8, wherein the looped handles are foldable into a knot whereby the mouth is made smaller thereby to form a tighter fit for the penis, and wherein the knot retains loops through which the elongated tying member can be threaded.

10. A collection bag for male incontinence comprising:
first and second opposing flexible side walls joined together and forming a fluid container for holding urine;
between the bag first and second side walls, a mouth is defined for receiving the penis and possible the scrotum;
handles for the bag; and
connectable through the handles and/or around the bag neck there is an anchoring for engaging the body.

11. A bag as claimed in claim 1, including a moisture management fabric composite having at least one of a first layer of said fabric composite, a relatively fine mesh fabric, a relatively open mesh fabric, and a yarn interconnecting the layers, a plurality of moisture-absorbent layers, hydrophilic fibers for receiving and dispersing moisture entering said fabric composite, and the absorbent layers being for trapping liquid moisture within said absorbent layers.

12. A bag as claimed in claim 11, including having the moisture management fabric comprise moisture wicking fibers adapted for promoting moisture flow by capillary wicking, the moisture wicking fibers being selectively polyester, nylon, or acrylic.

13. A bag as claimed in claim 11, including antimicrobial fibers, and wherein said moisture-absorbent fibers are selected from a group consisting of cotton and wool.

14. A bag as claimed in claim 11, wherein the absorbent layers comprise hydrophilic fibers selected from a group consisting of cotton, felt, and wool.

15. A method of assisting in collection of urine for male incontinence comprising:
locating a bag with flexible side walls to form a fluid container for holding urine such that a mouth of the bag receives the penis and possibly the scrotum; and
connecting handles for the bag and/or the bag neck with an anchoring for engaging the body.

16. A method as claimed in claim 15, including using a cord to surround the body torso, the cord being openable and fastenable, and selectively removable from the bag handles.

17. A method as claimed in claim 15, including placing in the bag a urine absorption pad, the pad including a moisture absorbing composite.

18. A kit for assisting in male incontinence comprising:
one or more bags each having flexible side walls to form a fluid container for holding urine;
between the walls there is a mouth defined for receiving the penis and possibly the scrotum;
handles for the bag; and
connected to the handles and/or around the bag neck there is an anchoring for engaging the body.

19. A kit as claimed in claim 18, including a cord to surround the body torso, the cord being openable and fastenable, and selectively removable from the bag handles.

20. A kit as claimed in claim 18, including a pad wherein when urine enters the bag, the urine is absorbed by the pad, the pad including a moisture absorbing fabric composite.
